(19) United States
(12) Patent Application Publication
(10) Pub. No.: US 2024/0164690 A1
(43) Pub. Date: May 23, 2024

(54) STATE ESTIMATION APPARATUS, STATE ESTIMATION METHOD AND PROGRAM

(71) Applicant: NIPPON TELEGRAPH AND TELEPHONE CORPORATION, Tokyo (JP)

(72) Inventors: Takayuki OGASAWARA, Musashino-shi (JP); Shingo TSUKADA, Musashino-shi (JP); Kentaro TANAKA, Musashino-shi (JP); Hiroshi NAKASHIMA, Musashino-shi (JP); Masumi YAMAGUCHI, Musashino-shi (JP); Toichiro GOTO, Musashino-shi (JP)

(73) Assignee: NIPPON TELEGRAPH AND TELEPHONE CORPORATION, Tokyo (JP)

(21) Appl. No.: 18/283,295

(22) PCT Filed: Mar. 26, 2021
(86) PCT No.: PCT/JP2021/012951

§ 371 Date: Sep. 21, 2023

(51) Int. Cl. A61B 5/352

(52) U.S. Cl. CPC A61B 5/352

(57) ABSTRACT

An aspect of the present invention is a state estimation device including a cardiac state time series acquisition unit that acquires a cardiac state time series that is a time series of a cardiac state quantity that is a quantity indicating a state of a heart as an estimation target, and a cardiac state estimation unit that estimates a state of the heart as the estimation target based on an occurrence time of out-of-range data, using, as the out-of-range data, a non-responsive period sample whose value is outside a threshold region of processing determined according to a distribution of the non-responsive period sample among non-responsive period samples that are non-responsive period samples among samples of the cardiac state time series acquired by the cardiac state time series acquisition unit.

### TECHNICAL FIELD

The present invention relates to a state estimation apparatus, a state estimation method and a program.

### BACKGROUND ART

In recent years, a biological signal, for example, a cardiac potential or a heart rate, can be easily used by an inexpensive and small wearable device (Non Patent Literature 1).

### CITATION LIST

### Non Patent Literature

Non Patent Literature 1: Kasai, Ogasawara, Nakashima, and Tsukada, “Development and practical use of functional material ‘hitoe’ that enables biological information measurement just by being worn” Communication Society Magazine No. 41, The Institute of Electronics, Information and Communication Engineers (June 2017) (Vol. 11 No. 1)
Non Patent Literature 2: “Jiji Medical, ventricular tachycardia and ventricular fibrillation”, [online], [retrieved on Feb. 10, 2021], Internet <https://medical.jiji.com/medical/012-1027>

### SUMMARY OF INVENTION

### Technical Problem

However, a means capable of detecting a heart disease with high reliability is limited to a medical device such as a large Holter electrocardiograph, and a wearable device has not been reliably achieved.

One of the reasons is the limitation of specifications of the wearable device. If it is attempted to achieve it by a small and inexpensive device, only limited arithmetic processing can be performed, and thus it is not possible to perform heart disease detection processing mounted on the Holter electrocardiograph. That is, since the amount of calculation required for processing of estimating the state of the heart such as whether or not an abnormality has occurred in the heart is large, a wearable device capable of estimating the abnormality of the heart has not been achieved. When an existing method with a large amount of calculation is used, a solution to reduce the amount of data to a capacity that can be implemented in a wearable device can also be assumed. However, since heart disease tends to occur suddenly in a short period of time, reducing the amount of data increases the likelihood of overlooking such rare trends. Then, as a consequence, there is a risk that it will not be possible to detect a serious life-threatening situation in an emergency. Therefore, for the benefit of the user, there is a need for a technique of reducing the amount of calculation required for estimation while not reducing a large amount of data sampled frequently.

In view of the above circumstances, an object of the present invention is to provide a technique for reducing the amount of calculation required for estimating the state of the heart.

### Solution to Problem

An aspect of the present invention is a state estimation device including a cardiac state time series acquisition unit that acquires a cardiac state time series that is a time series of a cardiac state quantity that is a quantity indicating a state of a heart as an estimation target, and a cardiac state estimation unit that estimates a state of the heart as the estimation target based on an occurrence time of out-of-range data, using, as the out-of-range data, a non-responsive period sample whose value is outside a threshold region of processing determined according to a distribution of the non-responsive period sample among non-responsive period samples that are non-responsive period samples among samples of the cardiac state time series acquired by the cardiac state time series acquisition unit.

An aspect of the present invention is a state estimation device including a cardiac state time series acquisition unit that acquires a cardiac state time series that is a time series of a cardiac state quantity that is a quantity indicating a state of a heart as an estimation target, and a cardiac state estimation unit that estimates a state of the heart as the estimation target based on a time interval RR-Interval (RRI) of an R wave in the cardiac state time series.

An aspect of the present invention is a state estimation method including a cardiac state time series acquisition step of acquiring a cardiac state time series that is a time series of a cardiac state quantity that is a quantity indicating a state of a heart as an estimation target, and a cardiac state estimation step of estimating a state of the heart as the estimation target based on an occurrence time of out-of-range data, using, as the out-of-range data, a non-responsive period sample whose value is outside a threshold region of processing determined according to a distribution of the non-responsive period sample among non-responsive period samples that are non-responsive period samples among samples of the cardiac state time series acquired in the cardiac state time series acquisition step.

According to still another aspect of the present invention, there is a program for causing a computer to function as the above-described state estimation device.

### Advantageous Effects of Invention

According to the present invention, it is possible to reduce the amount of calculation required for estimating the state of the heart.

### BRIEF DESCRIPTION OF DRAWINGS

FIG. 1 is an explanatory diagram for explaining an outline of an abnormal state estimation system 100 according to an embodiment.

FIG. 2 is a diagram illustrating an example of a cardiac state quantity time series obtained from the heart which is in a normal state in the embodiment.

FIG. 3 is a diagram illustrating an example of a cardiac state quantity time series obtained from the heart which is in an abnormal state according to the embodiment.

FIG. 4 is a diagram illustrating an upper threshold value, a lower threshold value, a threshold region, and out-of-range data according to the embodiment.

FIG. 5 is a diagram illustrating an example of a hardware configuration of a monitoring device 4 according to the embodiment.

FIG. 6 is a diagram illustrating an example of a functional configuration of a control unit 41 according to the embodiment.

FIG. 7 is a diagram illustrating an example of a hardware configuration of a control device 5 according to the embodiment.

FIG. 8 is a diagram illustrating an example of a functional configuration of a control unit 51 according to the embodiment.

FIG. 9 is a flowchart illustrating an example of a flow of processing executed by the abnormal state estimation system 100 according to the embodiment.

FIG. 10 is an explanatory diagram for explaining an effect obtained by fourth-type cardiac state estimation processing according to a modification.

### DESCRIPTION OF EMBODIMENTS

### Embodiment

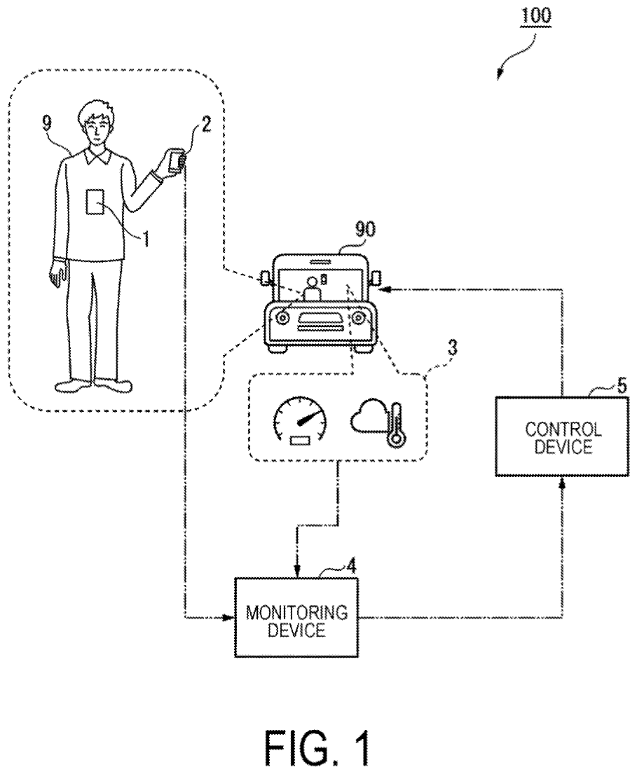

FIG. 1 is an explanatory diagram for explaining an outline of an abnormal state estimation system 100 according to an embodiment. The abnormal state estimation system 100 estimates an abnormality of the heart of an estimation target 9. The estimation target 9 may be any living body as long as the living body has a heart, and is, for example, a person. The estimation target 9 may be an animal other than a person. The estimation target 9 includes a biological signal acquisition device 1.

The biological signal acquisition device 1 acquires information (hereinafter referred to as a “cardiac state signal”) of a time series (hereinafter referred to as “cardiac state time series”) of an amount (hereinafter referred to as a “cardiac state quantity”) indicating the state of the heart of the estimation target 9 such as a time series of cardiac potential or a time series of heart rate. The biological signal acquisition device 1 is, for example, a device capable of acquiring a cardiac state signal, and is a wearable device worn by the estimation target 9. The biological signal acquisition device 1 is a device including a cardiac potential sensor that detects a cardiac potential from the estimation target 9 via, for example, conductive electrodes. The biological signal acquisition device 1 repeatedly acquires the cardiac state quantity of the estimation target. 9 at a predetermined time interval shorter than a unit processing period to be described later.

The abnormal state estimation system 100 estimates whether or not an abnormality has occurred in the heart of the estimation target 9 based on at least the cardiac state signal acquired by the biological signal acquisition device 1. For example, the abnormal state estimation system 100 estimates whether or not an abnormality has occurred in the heart for the estimation target 9 riding in an automobile 90. Hereinafter, for simplicity of description, the abnormal state estimation system 100 will be described by taking, as an example, a case of estimating an abnormality of the heart of the estimation target 9 riding in the automobile 90.

The abnormal state estimation system 100 includes a biological signal acquisition device 1, a relay terminal 2, an environment sensor 3, a monitoring device 4, and a control device 5. The biological signal acquisition device 1 outputs the acquired cardiac state signal to the relay terminal 2.

The relay terminal 2 is a device that transmits the cardiac state signal acquired by the biological signal acquisition device 1 to the monitoring device 4. The relay terminal 2 is, for example, a device including an antenna that transmits the cardiac state signal. The relay terminal 2 may be, for example, a portable terminal such as a smartphone or a tablet that acquires and transmits the cardiac state signal from the biological signal acquisition device 1.

The relay terminal 2 converts, for example, the cardiac state signal from an analog signal to a digital signal. Note that the cardiac state signal does not necessarily need to be transmitted from the relay terminal 2 in the form of a digital signal, and may be transmitted in the form of an analog signal. Note that the conversion of the cardiac state signal from the analog signal to the digital signal is not necessarily executed by the relay terminal 2, and may be executed by the biological signal acquisition device 1. Note that the conversion of the cardiac state signal from the analog signal to the digital signal may be executed by the monitoring device 4. For the sake of simplicity, the abnormal state estimation system 100 will be described by taking, as an example, a case where the cardiac state signal is transmitted from the relay terminal 2 in the form of a digital signal.

The environment sensor 3 is a sensor that acquires information (hereinafter referred to as “environment information”) regarding one or both of the motion state of the estimation target 9 and the environment in which the estimation target 9 exists. The environment sensor 3 is, for example, a speedometer that measures the moving speed of the estimation target 9. In such a case, the environment information indicates the moving speed of the estimation target 9. The environment sensor 3 may be, for example, a temperature sensor that measures the temperature of the space in which the estimation target 9 exists. In such a case, the environment information indicates the temperature of the space in which the estimation target 9 exists.

The environment sensor 3 may be, for example, an acceleration sensor that measures acceleration of movement of the estimation target 9. In such a case, the environment information indicates the acceleration of the movement of the estimation target 9. Note that the environment sensor 3 does not necessarily indicate only one type of information, and may indicate a plurality of types of information. For example, the environment sensor 3 may indicate the moving speed of the estimation target 9 and the temperature of the space in which the estimation target 9 exists.

The environment sensor 3 is, for example, a sensor mounted on the automobile 90, and may be a sensor that acquires information (hereinafter referred to as “in-vehicle information”) indicating the state of the automobile 90 such as an acceleration sensor, a temperature sensor, or a speedometer mounted on the automobile 90. The in-vehicle information is an example of the environment information.

The environment sensor 3 may be, for example, an acceleration sensor. Note that the environment sensor 3 is not necessarily mounted as a device different from the biological signal acquisition device 1, and may be provided in the biological signal acquisition device 1. The environment sensor 3 may be mounted as a device worn by the estimation target 9 or may be provided in the automobile 90 in which the estimation target 9 is riding.

The environment sensor 3 transmits the acquired environment information to the monitoring device 4.

The monitoring device 4 acquires the cardiac state signal and the environment information. The monitoring device 4 estimates the abnormality of the heart of the estimation target 9 based on at least the cardiac state signal. Hereinafter, processing in which the monitoring device 4 estimates the abnormality of the heart of the estimation target 9 based on at least the cardiac state signal will be referred to as cardiac state estimation processing. The cardiac state estimation processing is, for example, first-type cardiac state estimation processing to be described later.

Based on the estimation result of the monitoring device 4, the control device 5 determines whether or not an estimation result of the monitoring device 4 satisfies a notification criterion that is a predetermined criterion. Specifically, the notification criterion is a predetermined criterion for determining whether or not to notify a predetermined notification destination of the estimation result of the monitoring device 4 regarding the state of the heart of the estimation target 9. In a case where the estimation result satisfies the warning notification criterion, the control device 5 notifies the predetermined notification destination determined in advance that the heart of the estimation target 9 is abnormal. Hereinafter, processing of determining whether or not the estimation result of the monitoring device 4 satisfies the notification criterion will be referred to as notification determination processing.

<Description of First-Type Cardiac State Estimation Processing>

The first-type cardiac state estimation processing will be described. The first-type cardiac state estimation processing includes statistic calculation processing and abnormality estimation processing. The statistic calculation processing is repeatedly executed at a predetermined cycle. Hereinafter, a length of one cycle of the cycle in which the statistic calculation processing is executed will be referred to as a unit processing period. The length of the unit processing period is, for example, two seconds.

The statistic calculation processing is processing of calculating a statistic (hereinafter referred to as a “cardiac state statistic”) related to the cardiac state time series indicated by the cardiac state signal. The cardiac state statistic is, for example, a time average of the cardiac state quantity. The statistic of the cardiac state time series is, for example, a deviation of a distribution of the cardiac state quantity. The deviation may be any amount as long as the amount indicates a difference from an average value. Therefore, the deviation may be, for example, dispersion. The deviation may be, for example, a standard deviation.

In the statistic calculation processing, a statistic regarding the cardiac state time series is acquired using a sample satisfying a predetermined condition (hereinafter referred to as “sample condition”). The sample condition is, for example, a condition that all samples included in the cardiac state signal acquired by the monitoring device 4 during the unit processing period immediately before the statistic calculation processing is executed. Therefore, for example, if the unit processing period is two seconds, the number of samples used for the statistic calculation processing is all samples included in the cardiac state signal acquired by the monitoring device 4 in the last two seconds.

The abnormality estimation processing is processing of estimating whether or not the state of the heart of the estimation target 9 is in an abnormal state. The abnormality of the estimation target by the abnormality estimation processing is, for example, ventricular fibrillation. The abnormality estimation processing includes non-responsive period sample determination processing, out-of-range data determination processing, and ventricular abnormality determination processing.

For easy understanding of the non-responsive period sample determination processing, the out-of-range data determination processing, and the ventricular abnormality determination processing, a cardiac state quantity time series obtained from the heart in a normal state and a cardiac state quantity time series obtained from the heart in an abnormal state will be described.

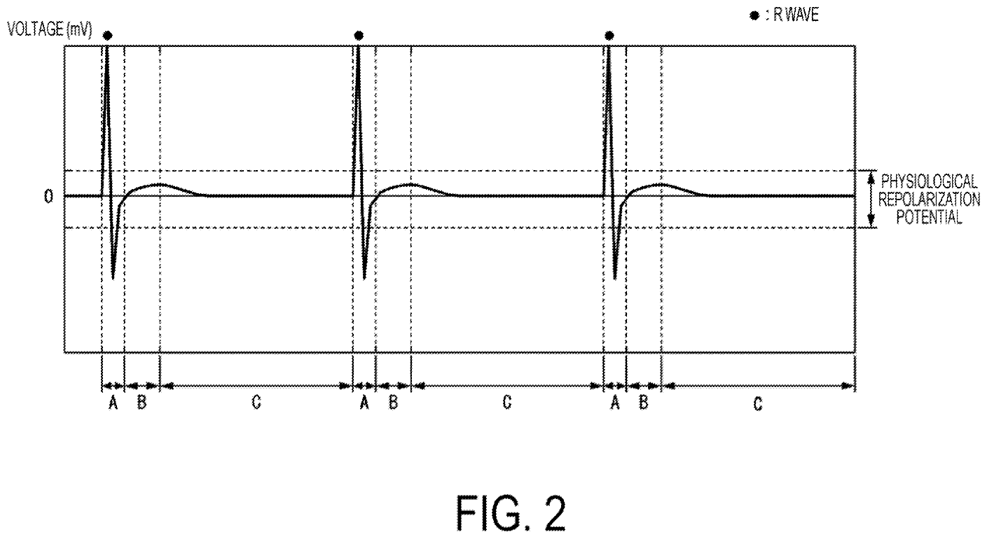

FIG. 2 is a diagram illustrating an example of a cardiac state quantity time series obtained from the heart which is in the normal state in the embodiment. More specifically, FIG. 2 is a diagram illustrating an example of a time series of cardiac potential obtained from the heart in the normal state. In FIG. 2, the vertical axis represents the potential of the cardiac potential, and the horizontal axis represents time.

When a normal heartbeat is performed, an electrocardiographic waveform including an R wave is observed. A black circle in FIG. 2 indicates an R wave. A, B, and C in FIG. 2 each indicate a type of an activity period related to polarization when the heart beats. Hereinafter, a period of the type A is referred to as an A period. Hereinafter, a period of the type B is referred to as a B period. Hereinafter, a period of the type C is referred to as a C period.

The period A is a polarization section of the myocardium. In the period A, an R waveform is mainly observed. The period B is an absolute non-responsive period. The period B is a period immediately after the myocardial polarization. In the period B, in the principle of the myocardium, when the state of the heart is normal, there is no generation of the cardiac potential corresponding to the waveform. The period C is a relative non-responsive period. In the period C, if the state of the heart is normal, there is no waveform due to a pulsatile trend of a constant rhythm. In other words, if the state of the heart is normal, the polarization repeats periodically, but since the C period is a period between the repeated polarization and polarization, there is no waveform. Note that, in a case where the period B and the period C are not distinguished from each other, they are generally called non-responsive periods.

As described above, in the case of the time series of the cardiac potential obtained from the normal heart, the A period, the B period, and the C period can be determined. In addition, in a case of the time series of the cardiac potential obtained from the normal heart, a voltage change from 0 millivolts is smaller in the non-responsive period of the cardiac potential (that is, the B period and the C period) than in the A period in which the R wave is generated. The range of the voltage change in the A period in time series of the cardiac potential obtained from the normal heart is generally referred to as a range of a physiologically normal repolarization potential change.

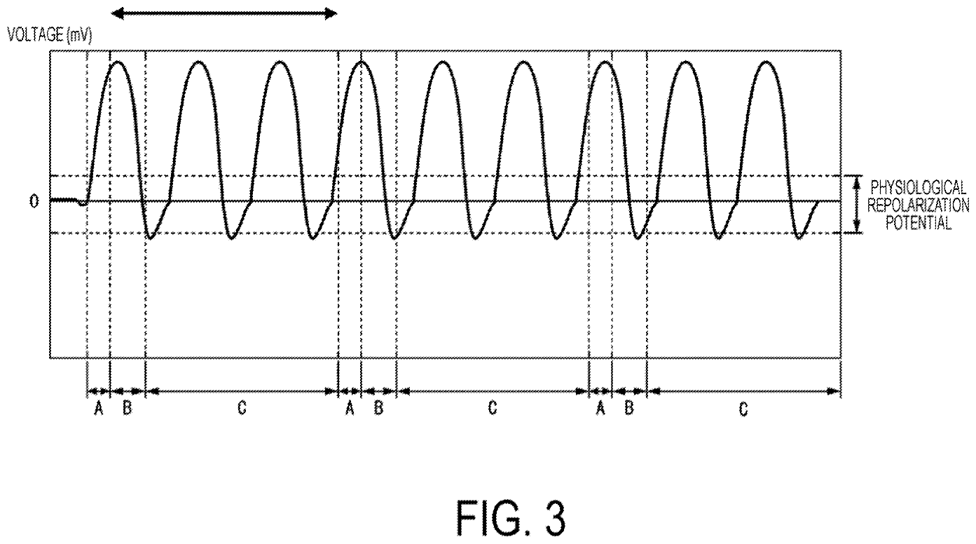

FIG. 3 is a diagram illustrating an example of a cardiac state quantity time series obtained from the heart which is in an abnormal state according to the embodiment. Specifically, FIG. 3 is a diagram illustrating an example of a time series of the cardiac potential obtained from the heart which is in the abnormal state. More specifically, FIG. 3 is a diagram illustrating an example of a time series of cardiac potential obtained from the heart in a state of ventricular fibrillation. In FIG. 3, the vertical axis represents the potential of the cardiac potential, and the horizontal axis represents time.

A, B, and C in FIG. 3 indicate the A period, the B period, and the C period, respectively. FIG. 3 illustrates that, in the cardiac potential during ventricular fibrillation, the behavior of the cardiac potential out of the range of the physiologically normal repolarization potential change occurs even in the period corresponding to the non-responsive period (that is, the period B and the period C) in the normal cardiac potential.

The abnormal state estimation system 100 is a system that estimates whether the state of the heart of the estimation target 9 is normal or abnormal based on a difference in such behavior of the cardiac potential existing between the normal heart and the abnormal heart. The out-of-range data determination processing executed in the abnormal state estimation system 100 is processing executed to quantify the degree of generation of out-of-range data in the section corresponding to the non-responsive period of the normal cardiac potential using the statistic.

Each of the non-responsive period sample determination processing, the out-of-range data determination processing, and the ventricular abnormality determination processing will be described.

The non-responsive period sample determination processing is processing of determining which sample belongs to the non-responsive period among samples of the cardiac state time series. The non-responsive period sample determination processing is, for example, processing of determining a sample satisfying a predetermined condition as a sample belonging to the non-responsive period.

The predetermined condition is, for example, a condition that the sample exceeds a predetermined threshold value. The threshold value is specifically a statistic of the cardiac state time series within a predetermined section. The statistic is, for example, a sum of a predetermined representative value and a predetermined dispersion degree. The statistic may be, for example, a difference between a predetermined representative value and a predetermined dispersion degree. The representative value is, for example, an average value. The dispersion degree is, for example, a standard deviation.

However, samples of the cardiac state time series may momentarily exceed a threshold value. Therefore, in the non-responsive period sample determination processing, it may be determined whether or not the sample satisfies a predetermined condition with respect to the number of times the sample continuously crosses the threshold value. In the non-responsive period sample determination processing, when a predetermined condition for the number of times the sample continuously crosses the threshold value is satisfied, it is determined that the sample belongs to the non-responsive period.

The threshold value may be, for example, a cardiac state statistic acquired by statistic calculation processing.

In order to simplify the description below, the abnormal state estimation system 100 will be described by taking, as an example, a case where the non-responsive period sample determination processing is processing of determining which sample belongs to the non-responsive period among the samples of the cardiac state time series based on the cardiac state statistic acquired by the statistic calculation processing. Note that, in a case where the non-responsive period sample determination processing is processing of determining a sample satisfying a predetermined condition as a sample belonging to the non-responsive period, the statistic calculation processing does not necessarily need to be executed.

The out-of-range data determination processing is executed for the sample (hereinafter referred to as a “non-responsive period sample”) determined to be a sample belonging to the non-responsive period by the non-responsive period sample determination processing.

The out-of-range data determination processing is processing of determining whether or not the value of each non-responsive period sample is out of the range (hereinafter referred to as a “threshold region”) corresponding to each time position. The time position is a position in a time axis direction of each of the samples of the cardiac state time series. Hereinafter, the non-responsive period sample whose value (that is, the cardiac state quantity) is determined to be out of the range of the threshold region by the out-of-range data determination processing is referred to as out-of-range data.

The threshold region is a range having at least an upper limit value and a lower limit value. The upper limit value of the threshold region is hereinafter referred to as an upper threshold value. The lower limit value of the threshold region is hereinafter referred to as a lower threshold value.

The threshold region is determined for each unit processing period according to the distribution of the non-responsive period sample within the unit processing period. The upper threshold value is, for example, (M+V) when the average value of the cardiac state quantity indicated by the non-responsive period sample within the unit processing period including the time position where the threshold region is determined is M and the standard deviation is V. The lower threshold value is, for example, (M−V) when the average value of the cardiac state quantity indicated by the non-responsive period sample within the unit processing period is M and the standard deviation is V.

Note that the upper threshold value and the lower threshold value are not necessarily limited to the sum or difference of the average value M and the standard deviation V. The upper threshold value and the lower threshold value may be a sum or a difference of values obtained by multiplying the standard deviation V by a constant (correction value) and performing adjustment according to detection sensitivity. The upper threshold value and the lower threshold value may be a result of conversion by a predetermined function using the average value M and the standard deviation V as independent variables.

In addition, the upper threshold value and the lower threshold value may be calculated on the basis of the variance or gradient of the cardiac state quantity. The upper threshold value and the lower threshold value may be calculated based on an amount of adjustment based on a device other than the biological signal, environmental data, or continuity (presence or absence of missing of the observation value). Being outside the range of the threshold region means that the value is either less than the lower threshold value or more than the upper threshold value.

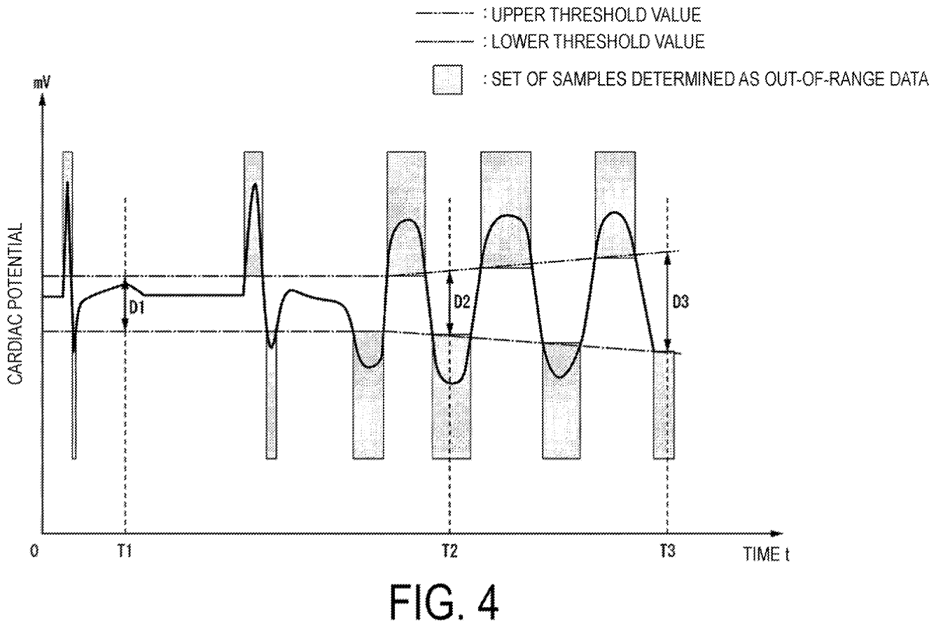

FIG. 4 is a diagram illustrating the upper threshold value, the lower threshold value, the threshold region, and the out-of-range data according to the embodiment. FIG. 4 illustrates cardiac potential time series as an example of cardiac state time series. The horizontal axis in FIG. 4 indicates the elapsed time from the time of the origin. In FIG. 4, the vertical axis represents the cardiac potential. FIG. 4 illustrates the upper threshold value and the lower threshold value. The upper threshold value and the lower threshold value in the example of FIG. 4 are examples of values calculated using cardiac potential data for the last two seconds. Therefore, as illustrated in FIG. 4, the upper threshold value and the lower threshold value are not necessarily the same at all times.

In FIG. 4, the ranges of the cardiac potential indicated by D1, D2, and D3 are threshold regions at time T1, time T2, and time T3, respectively. As illustrated in FIG. 4, the range of the cardiac potential indicated by the threshold region is not necessarily the same at all times. FIG. 4 illustrates a set of non-responsive period samples determined to be out-of-range data.

The ventricular abnormality determination processing is processing of estimating the state of the ventricle based on the sample determined to be the out-of-range data by the out-of-range data determination processing. The ventricular abnormality determination processing is processing of determining that the ventricular state is abnormal when a condition indicating a predetermined appearance manner of a peak period and indicating an appearance manner of the peak period in a case where the ventricular state is an abnormal state (hereinafter referred to as a “peak period appearance condition”) is satisfied.

The peak period is a peak determination target period in which the out-of-range data integration time exceeds the threshold time. The out-of-range data integration time is a value obtained for each peak determination target period. The out-of-range data integration time is a value of integration of occurrence times of samples determined to be out-of-range data by the out-of-range data determination processing among the samples in each peak determination target period. That is, the out-of-range data integration time is a result obtained by adding a predetermined time width to each sample and multiplying the time width by the number of samples determined to be the out-of-range data among the samples within each peak determination target period.

The peak determination target period is a period having a predetermined length. The start time of the peak determination target period is a time that satisfies a predetermined condition. The start time of the peak determination target period is, for example, the end time of the immediately preceding peak determination target period. The start time of the peak determination target period may be, for example, a condition that the start time is a time when a predetermined time has elapsed from the immediately preceding peak determination target period.

The condition that it is a time when the predetermined time has elapsed from the immediately preceding peak determination target period means that the peak determination target period is periodically set in the ventricular abnormality determination processing. In the ventricular abnormality determination processing, for example, first, it is determined whether or not 0 milliseconds to 200 milliseconds of the cardiac state time series are set as the peak determination target periods and are peak periods. In the ventricular abnormality determination processing, next, processing of setting a period having a length of 200 milliseconds subsequent to the time of 200 milliseconds being set as new 0 milliseconds as a new peak determination target period is repeated.

The threshold time is a predetermined reference time and is a reference time for detecting a value that does not occur in the cardiac state time series of the normal heart. More specifically, the threshold time is a predetermined reference time that is longer than the out-of-range data integration time in the cardiac state time series of the normal heart. Since the threshold time is longer than the out-of-range data integration time in the cardiac state time series of the normal heart, the peak determination target period in which the out-of-range data integration time exceeds the threshold time is a period in which the cardiac state time series of the abnormal heart appears.

The length of the peak period is 15 milliseconds, which is three times five milliseconds, for example, in a case where the cardiac state time series is acquired at a sampling rate of 200 Hz and three points are out-of-range data. Note that the time interval of each sample in the time series at the sampling rate of 200 Hz is five milliseconds. In a case where the cardiac state time series is acquired at a sampling rate of 200 Hz, the occurrence time of the sample, that is, the predetermined time width added to the sample, would be, for example, five milliseconds.

The length of the peak determination target period is desirably substantially the same as the length of one beat of the heartbeat. Therefore, the length of the peak determination target period is, for example, 200 milliseconds.

The threshold time is, for example, a time longer than the occurrence time of the R wave of the normal heart. The time longer than the occurrence time of the R wave of the normal heart is, for example, 50 milliseconds.

An example of processing executed in the ventricular abnormality determination processing in a case where the threshold time is 50 milliseconds, the length of the peak determination target period is 200 milliseconds, and the peak determination target period is periodically set every 200 milliseconds will be specifically described. In this case, in the ventricular abnormality determination processing, processing of determining, as a peak period, there is executed a peak determination target period in which a cumulative time of the non-responsive period sample in which the cardiac state quantity is determined to be outside the threshold region is 50 milliseconds or more among the peak determination target periods that are periodically repeated at intervals of 200 milliseconds.

The peak period appearance condition is, for example, a condition that a predetermined number of peak periods continuously appear. In a case where the peak period appearance condition is a condition that a predetermined number of peak periods continuously appear, the heart of the estimation target 9 is in a state where ventricular flutter or ventricular fibrillation has occurred. The number of consecutive peak periods is a predetermined value set in advance, but is desirably a value determined in consideration of, for example, a balance between the frequency of erroneous determination and the time required to obtain a determination result.

More specifically, the value is desirably a value that achieves both the low frequency of erroneous determination and the short time required to obtain the determination result. The time required to obtain the determination result is desirably, for example, a time in which damage that may occur when the heart of the estimation target 9 is in an abnormal state can be prevented by notification. The number of consecutive peak periods is, for example, five.

As described above, the first-type cardiac state estimation processing is processing of estimating the state of the heart of the estimation target 9 based on the occurrence time of the out-of-range data.

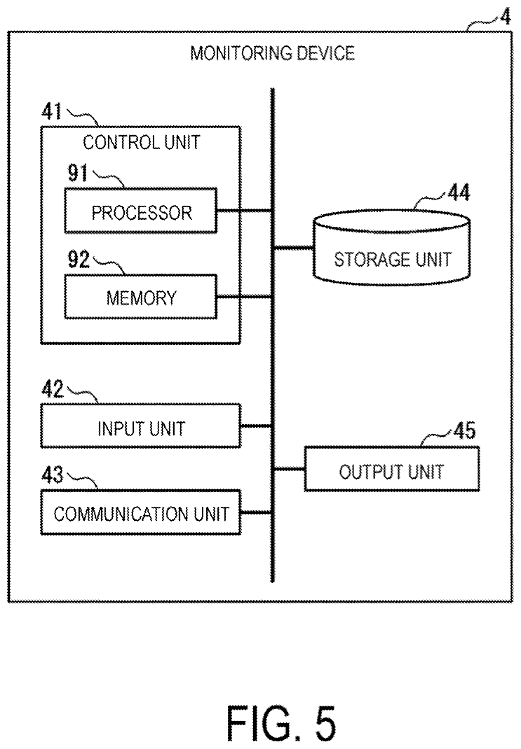

FIG. 5 is a diagram illustrating an example of a hardware configuration of the monitoring device 4 according to the embodiment. The monitoring device 4 includes a control unit 41 including a processor 91 such as a central processing unit (CPU) and a memory 92, which are connected by a bus, and executes a program. The monitoring device 4 functions as a device including the control unit 41, an input unit 42, a communication unit 43, a storage unit 44, and an output unit 45 by executing the program.

More specifically, the processor 91 reads the program stored in the storage unit 44, and stores the read program in the memory 92. The processor 91 executes the program stored in the memory 92, whereby the monitoring device 4 functions as a device including the control unit 41, the input unit 42, the communication unit 43, the storage unit 44, and the output unit 45.

The control unit 41 controls operations of various functional units included in the monitoring device 4. The control unit 41 executes, for example, the cardiac state estimation processing. The control unit 41 controls, for example, the operation of the output unit 45. The control unit 41 records, for example, various types of information generated by execution of the cardiac state estimation processing in the storage unit 44. The control unit 41 records, for example, the cardiac state time series indicated by the cardiac state signal input to the input unit 42 or the communication unit 43 in the storage unit 44.

The input unit 42 includes an input device such as a mouse, a keyboard, or a touch panel. The input unit 42 may be configured as an interface that connects these input devices to the monitoring device 4. The input unit 42 receives inputs of various types of information to the monitoring device 4. For example, a cardiac state signal is input to the input unit 42. For example, the environment information may be input to the input unit 42.

The communication unit 43 includes a communication interface for connecting the monitoring device 4 to an external device. The communication unit 43 communicates with an external device in a wired or wireless manner. The external device is, for example, a device from which a cardiac state signal is transmitted. The transmission source of the cardiac state signal is, for example, the relay terminal 2. The external device is, for example, the control device 5. The communication unit 43 may communicate with the environment sensor 3. In a case where the communication unit 43 communicates with the environment sensor 3, the communication unit 43 may acquire the environment information acquired by the environment sensor 3 by communication with the environment sensor 3.

The storage unit 44 is configured using a computer-readable storage medium device such as a magnetic hard disk device or a semiconductor storage device. The storage unit 44 stores various types of information regarding the monitoring device 4. The storage unit 44 stores, for example, information input via the input unit 42 or the communication unit 43. The storage unit 44 stores, for example, various types of information generated by execution of the cardiac state estimation processing.

Note that the cardiac state signal and the environment information do not necessarily need to be input only to the input unit 42, and do not need to be input only to the communication unit 43. The cardiac state signal and the environment information may be input from either the input unit 42 or the communication unit 43.

The output unit 45 outputs various types of information. The output unit 45 includes a display device such as a cathode ray tube (CRT) display, a liquid crystal display, or an organic electro-luminescence (EL) display, for example. The output unit 45 may be configured as an interface that connects these display devices to the monitoring device 4. The output unit 45 outputs, for example, information input to the input unit 42. The output unit 45 may display, for example, an execution result of the cardiac state estimation processing.

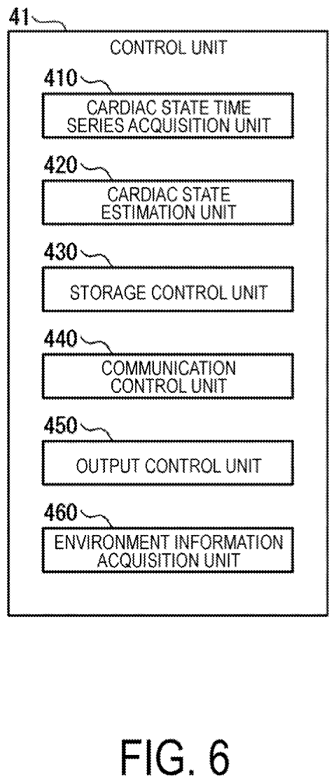

FIG. 6 is a diagram illustrating an example of a functional configuration of the control unit 41 according to the embodiment. The control unit 41 includes a cardiac state time series acquisition unit 410, a cardiac state estimation unit 420, a storage control unit 430, a communication control unit 440, an output control unit 450, and an environment information acquisition unit 460.

The cardiac state time series acquisition unit 410 repeatedly acquires a cardiac state time series signal at a predetermined cycle via the input unit 42 or the communication unit 43. That is, the cardiac state time series acquisition unit 410 acquires the cardiac state time series.

The cardiac state estimation unit 420 estimates the state of the heart of the estimation target 9 based on the cardiac state time series indicated by the cardiac state signal acquired by the cardiac state time series acquisition unit 410. The cardiac state estimation unit 420 estimates the state of the heart of the estimation target 9 by executing cardiac state estimation processing on the cardiac state time series indicated by the cardiac state signal acquired by the cardiac state time series acquisition unit 410, for example. The cardiac state estimation processing executed by the cardiac state estimation unit 420 is, for example, first-type cardiac state estimation processing.

The storage control unit 430 records various types of information in the storage unit 44. The communication control unit 440 controls the operation of the communication unit 43. The communication control unit 440 controls the operation of the communication unit 43 to cause the communication unit 43 to transmit, for example, the estimation result of the cardiac state estimation unit 420 to the control device 5. The output control unit 450 controls the operation of the output unit 45. For example, the output control unit 450 controls the operation of the output unit 45 to cause the output unit 45 to output the estimation result of the cardiac state estimation unit 420.

The environment information acquisition unit 460 repeatedly acquires environment information at a predetermined cycle via the input unit 42 or the communication unit 43. That is, the cardiac state time series acquisition unit 410 acquires the environment information.

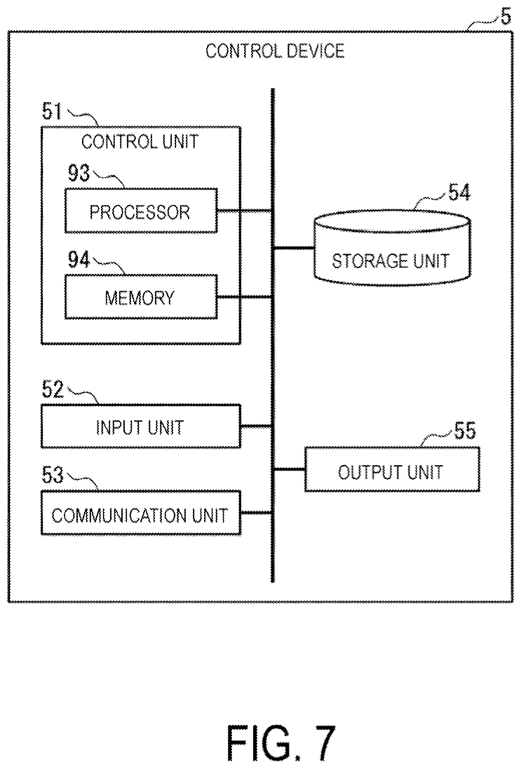

FIG. 7 is a diagram illustrating an example of a hardware configuration of the control device 5 according to the embodiment. The control device 5 includes a control unit 51 including a processor 93 such as a CPU and a memory 94 connected by a bus, and executes a program. The control device 5 functions as a device including a control unit 51, an input unit 52, a communication unit 53, a storage unit 54, and an output unit 55 by executing a program.

More specifically, the processor 93 reads the program stored in the storage unit 44, and stores the read program in the memory 94. The processor 93 executes the program stored in the memory 94, whereby the control device 5 functions as a device including the control unit 51, the input unit 52, the communication unit 53, the storage unit 54, and the output unit 55.

The control unit 51 controls operations of various functional units included in the control device 5. The control unit 51 executes, for example, the notification determination processing. The control unit 51 controls, for example, the operation of the communication unit 53. The control unit 51 controls, for example, the operation of the communication unit 53 to transmit the notification to the notification destination. The control unit 51 controls, for example, the operation of the output unit 55. The control unit 51 records, for example, various types of information generated by execution of the notification determination processing in the storage unit 54. The control unit 51 records, for example, information input to the input unit 52 or the communication unit 53 in the storage unit 54. The information input to the input unit 52 or the communication unit 53 is, for example, an estimation result of the cardiac state estimation unit 420.

The input unit 52 includes an input device such as a mouse, a keyboard, or a touch panel. The input unit 52 may be configured as an interface that connects these input devices to the control device 5. The input unit 52 receives inputs of various types of information to the control device 5. For example, an estimation result of the cardiac state estimation unit 420 is input to the input unit 52.

The communication unit 53 includes a communication interface for connecting the control device 5 to an external device. The communication unit 53 communicates with an external device in a wired or wireless manner. The external device is, for example, the monitoring device 4. The external device is, for example, a predetermined notification destination determined in advance.

The storage unit 54 is configured using a computer-readable storage medium device such as a magnetic hard disk device or a semiconductor storage device. The storage unit 54 stores various types of information regarding the control device 5. The storage unit 54 stores, for example, information input via the input unit 52 or the communication unit 53. The storage unit 54 stores, for example, various types of information generated by execution of the notification determination processing.

Note that the estimation result of the cardiac state estimation unit 420 (that is, the estimation result of the monitoring device 4) does not necessarily need to be input only to the input unit 52, and does not need to be input only to the communication unit 53. The estimation result of the cardiac state estimation unit 420 may be input from either the input unit 52 or the communication unit 53.

The output unit 55 outputs various types of information. The output unit 55 includes, for example, a display device such as a CRT display, a liquid crystal display, or an organic EL display. The output unit 55 may be configured as an interface that connects these display devices to the control device 5. The output unit 55 outputs, for example, information input to the input unit 52. The output unit 55 may display, for example, an estimation result input to the input unit 52 or the communication unit 53. The output unit 55 may display, for example, an execution result of the notification determination processing.

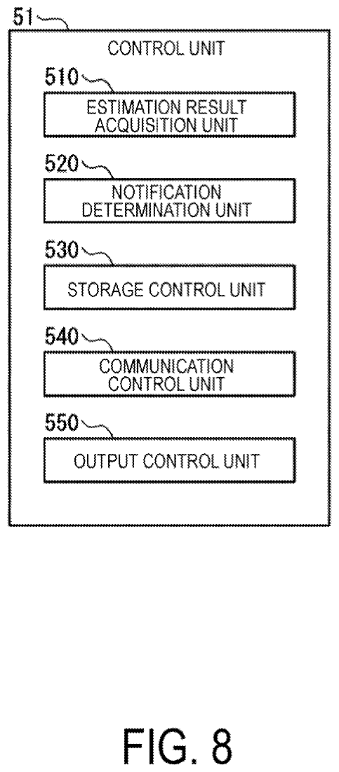

FIG. 8 is a diagram illustrating an example of a functional configuration of the control unit 51 according to the embodiment. The control unit 51 includes an estimation result acquisition unit 510, a notification determination unit 520, a storage control unit 530, a communication control unit 540, and an output control unit 550.

The estimation result acquisition unit 510 repeatedly acquires the estimation result of the cardiac state estimation unit 420 input to the input unit 52 or the communication unit 53 at a predetermined cycle.

The notification determination unit 520 executes the notification determination processing on the estimation result acquired by the estimation result acquisition unit 510. That is, the notification determination unit 520 determines whether or not the estimation result acquired by the estimation result acquisition unit 510 satisfies the notification criterion.

The storage control unit 530 records various types of information in the storage unit 54. The communication control unit 540 controls the operation of the communication unit 53.

The communication control unit 540 controls the operation of the communication unit 53 to cause the communication unit 53 to execute, for example, notification to a notification destination. The communication control unit 540 may cause the communication unit 53 to transmit a control signal for controlling the operation of the automobile 90, such as a signal for instructing the automobile 90 to decelerate or a signal for instructing the automobile 90 to stop.

The output control unit 550 controls the operation of the output unit 55. For example, the output control unit 550 controls the operation of the output unit 55 to cause the output unit 55 to output the determination result of the notification determination unit 520.

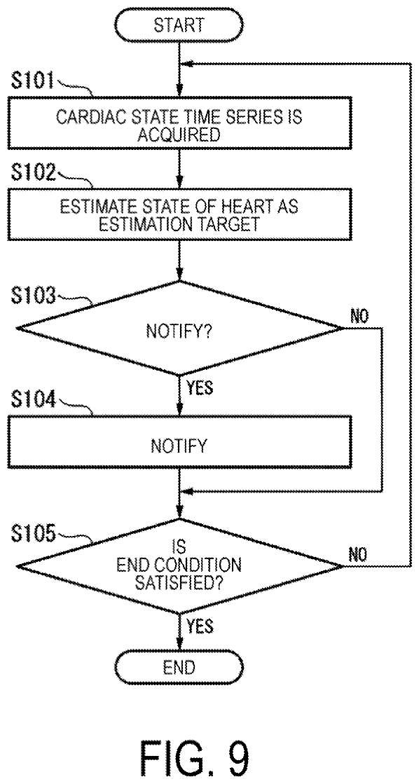

FIG. 9 is a flowchart illustrating an example of a flow of processing executed by the abnormal state estimation system 100 according to the embodiment. The abnormal state estimation system 100 repeatedly executes the processing illustrated in the flowchart illustrated in FIG. 9 until a predetermined end condition is satisfied. The predetermined end condition is, for example, a condition that power supply to the biological signal acquisition device 1 is interrupted. For example, the control unit 41 determines whether or not the end condition is satisfied. For example, the cardiac state estimation unit 420 determines whether or not the end condition is satisfied. For example, the notification determination unit 520 may determine whether or not the end condition is satisfied.

The cardiac state time series acquisition unit 410 acquires the cardiac state time series acquired from the estimation target 9 (step S101). Next, the cardiac state estimation unit 420 estimates the state of the heart of the estimation target 9 based on the cardiac state time series acquired in step S101 (step S102). Next, the notification determination unit 520 determines whether or not to notify the notification destination based on the estimation result of step 3102 (step S103).

When it is determined to notify the notification destination (step S103: YES), the communication control unit 540 controls the operation of the communication unit 53 to notify the notification destination (step S104). After step 3104, it is determined whether or not the end condition is satisfied (step S105). When the end condition is satisfied (step S105: YES), the processing ends. When the end condition is not satisfied (step 3105: NO), the processing returns to step 3101.

In a case where it is determined not to notify the notification destination (step S103: NO), the processing of step S105 is executed.

The abnormal state estimation system 100 according to the embodiment configured as described above estimates the state of the heart of the estimation target 9 only by processing with a small amount of calculation such as processing of calculating a statistic such as an average or a deviation, processing of determining whether or not a threshold value is exceeded, and processing of counting a period, the number of times, and the like. Therefore, the abnormal state estimation system 100 can reduce the amount of calculation required for estimating the state of the heart.

Further, the abnormal state estimation system 100 does not determine whether or not the data is out-of-range data for all the samples of the cardiac state time series, but determines whether or not the data is out-of-range data for the samples of the non-responsive period. Therefore, the abnormal state estimation system 100 can reduce the calculation amount required for estimating the abnormality of the heart as compared with a case where the determination as to whether or not the data is out-of-range data is performed for all the samples of the cardiac state time series. Further, the abnormal state estimation system 100 can perform highly accurate estimation that prevents erroneous determination by avoiding determination in a polarization section in which a normal waveform occurs.

Further, the abnormal state estimation system 100 includes the notification determination unit 520 and the communication control unit 540 to have a function of notifying a notification destination. Since having a notification function, the abnormal state estimation system 100 can appeal an action such as a call or an alarm to a driver of the automobile 90 such as a bus driver as necessary (that is, according to the determination of the notification determination unit 520). Therefore, the abnormal state estimation system 100 can reduce the risk caused by the abnormal state of the heart of the estimation target 9.

Further, since the abnormal state estimation system 100 includes the notification determination unit 520 and the communication control unit 540, it is also possible to transmit a control signal for causing the automobile 90 to decelerate or stop directly, not to the driver. Therefore, the abnormal state estimation system 100 can reduce the risk caused by the abnormal state of the heart of the estimation target 9.

(First Modification)

As the cardiac state estimation processing to be executed, the cardiac state estimation unit 420 may execute second-type cardiac state estimation processing instead of the first-type cardiac state estimation processing. That is, the cardiac state estimation unit 420 may estimate the state of the heart of the estimation target 9 by executing the second-type cardiac state estimation processing on the cardiac state time series indicated by the cardiac state signal acquired by the cardiac state time series acquisition unit 410.

<Second-Type Cardiac State Estimation Processing>

The second-type cardiac state estimation processing is processing of estimating the state of the heart of the estimation target 9 based on a time interval RRI (PR-Interval) of the R wave in the cardiac state time series.

The second-type cardiac state estimation processing is processing of estimating that the state of the heart of the estimation target 9 is abnormal, for example, when RRI in the cardiac state time series is smaller than an RRI lower limit threshold value that is a predetermined threshold value. The RRI lower limit threshold value is, for example, RRI during exercise of a person whose state of the heart is normal. The value larger than the RRI during exercise of a person whose state of the heart is normal is, for example, 600 ms.

In a case where the RRI lower limit threshold value is the RRI during exercise of a person whose state of the heart is normal, when the RRI of the cardiac state time series is smaller than the RRI lower limit threshold value, the possibility of occurrence of ventricular tachycardia is high. Therefore, by estimating the state of the heart depending on whether or not RRI in the cardiac state time series is smaller than the RRI lower limit threshold value, it is possible to estimate whether or not the heart of the estimation target 9 is in an abnormal state in which ventricular tachycardia occurs.

In the second-type cardiac state estimation processing, when RRI in the cardiac state time series is larger than an RRI upper limit threshold value which is a predetermined threshold value different from the RRI lower limit threshold value, it may be estimated that the state of the heart of the estimation target 9 is abnormal. When the condition of the heart is abnormal, the activity of the heart may decrease and the pulse rate may decrease. That is, when the state of the heart is abnormal, bradycardia may occur.

By estimating the state of the heart depending on whether or not RRI in the cardiac state time series is larger than the RRI upper limit threshold value, it is possible to estimate whether or not the heart of the estimation target 9 is in an abnormal state in which bradycardia occurs. The RRI upper limit threshold value is preferably a value with which the occurrence of bradycardia can be estimated, and is desirably, for example, 1000 ms or more.

In the second-type cardiac state estimation processing, the state of the heart of the estimation target 9 may be estimated using the RRI lower limit threshold value and the PRI upper limit threshold value.

The second-type cardiac state estimation processing may be processing of estimating the state of the heart of the estimation target 9 by further using environment information. The environment information used by the second-type cardiac state estimation processing for estimating the state of the heart of the estimation target 9 is, for example, information acquired by an inertial sensor such as an acceleration sensor or a gyro sensor, and is information indicating acceleration of the estimation target 9 (hereinafter referred to as “detection target acceleration information”). That is, in a case where the second-type cardiac state estimation processing uses the environment information for the state of the heart of the estimation target 9, the environment sensor 3 as a source of the environment information is, for example, an inertial sensor.

Even when the state of the heart of the estimation target 9 is not in the state where the ventricular tachycardia occurs and is normal, the RRI decreases when the estimation target 9 performs exercise. Therefore, the second-type cardiac state estimation processing using not only the cardiac state time series but also the detection target acceleration information can estimate the state of the heart of the estimation target 9 with higher accuracy than the second-type cardiac state estimation processing based only on the cardiac state time series.

In the second-type cardiac state estimation processing using not only the cardiac state time series but also the detection target acceleration information, normality determination is performed. The normality determination is processing of determining that the state of the heart of the estimation target 9 is normal in a case where the statistic obtained from the detection target acceleration information exceeds a threshold value satisfying a predetermined condition even in a case where the RRI obtained from the cardiac state time series becomes smaller than a predetermined reference.

Hereinafter, the predetermined condition satisfied by the threshold value in the normal determination is referred to as a normality threshold condition. The normality threshold condition is, for example, a condition of a predetermined value determined in advance. In such a case, the threshold value satisfying the normality threshold condition is a predetermined value determined in advance. In the normality determination, the state of the heart of the estimation target 9 is determined to be abnormal only in a case where the RRI obtained from the cardiac state time series becomes smaller than the predetermined standard and the statistic calculated based on the detection target acceleration information does not exceed the threshold value satisfying the normality threshold condition.

This is because, even when the RRI decreases, in a case where the statistic obtained from the detection target acceleration information is large, there is a high possibility that the decrease in RRI was caused by movement or exercise of the estimation target 9 rather than by the state abnormality of the heart.

Note that the statistic obtained from the detection target acceleration information is specifically information acquired by the inertial sensor, and is a statistic of distribution of values of each sample indicated by a time series of information indicating the acceleration of the estimation target 9.

Note that the statistic in the normality determination is, for example, a value obtained by integrating absolute values of acceleration values of three axes in a predetermined constant time. However, the statistic in the normality determination may be any value as long as the statistic is a statistic calculated based on the detection target acceleration information, and is not limited to a value obtained by integrating absolute values of acceleration values of the three axes in a predetermined constant time.

The normality threshold condition is not necessarily a condition of a predetermined value determined in advance. The normality threshold condition may be, for example, a statistic calculated based on the detection target acceleration information in the past time section satisfying a predetermined condition regarding the period (hereinafter referred to as a “normality determination period condition”). The normality determination period condition is, for example, a condition of three seconds before.

The normality threshold condition may be, for example, a condition that the normality threshold condition is a value of an objective variable of a predetermined function having the detection target acceleration information in the past time section satisfying the normality determination period condition as an explanatory variable.

The environment information used by the second-type cardiac state estimation processing for estimation of the state of the heart of the estimation target 9 may include, for example, position information of the estimation target 9. The position information of the estimation target 9 is, for example, information acquired using a technology for acquiring position information such as a global positioning system (GPS). That is, the environment sensor 3 that acquires the position information is a device that acquires the position information of the estimation target 9 using a technology of acquiring position information such as GPS of a smartphone or the like equipped with a GPS function, for example.

When the estimation target 9 is driving the automobile 90, strenuous exercise is not often performed. Therefore, the decrease in RRI when the information indicating that the estimation target 9 is on the roadway or the information indicating that the estimation target 9 is moving at a speed equal to or higher than the walking speed such as 50 km/h is acquired based on the position information is not the decrease in RRI caused by the exercise of the estimation target 9.

Therefore, the decrease in RRI when the information indicating that the estimation target 9 is on the roadway or the information indicating that the estimation target 9 is moving at a speed equal to or higher than the walking speed such as 50 km/h is acquired based on the position information means that the probability that the state of the heart of the estimation target 9 is abnormal is high. Therefore, in a case where the second-type cardiac state estimation processing estimates the state of the heart of the estimation target 9 also based on the position information, the state of the heart of the estimation target 9 can be estimated with higher accuracy than in a case where the state of the heart of the estimation target 9 is estimated without using the position information.

The abnormal state estimation system 100 of the first modification configured as described above estimates the state of the heart of the estimation target 9 only by processing with a small amount of calculation such as processing of calculating a statistic such as an average or a deviation, processing of determining whether or not a threshold value is exceeded, and processing of counting a period, the number of times, and the like. Therefore, the abnormal state estimation system 100 can reduce the amount of calculation required for estimating the abnormality of the heart.

(Second Modification)

As the cardiac state estimation processing to be executed, the cardiac state estimation unit 420 may execute third-type cardiac state estimation processing instead of the first-type cardiac state estimation processing. That is, the cardiac state estimation unit 420 may estimate the state of the heart of the estimation target 9 by executing the third-type cardiac state estimation processing on the cardiac state time series indicated by the cardiac state signal acquired by the cardiac state time series acquisition unit 410.

<Third-Type Cardiac State Estimation Processing>

The third-type cardiac state estimation processing is processing of executing the first-type cardiac state estimation processing, the second-type cardiac state estimation processing, and first integrated estimation processing. The first integrated estimation processing is processing of estimating the state of the heart of the estimation target 9 based on the estimation result of the first-type cardiac state estimation processing and the estimation result of the second-type cardiac state estimation processing.

In ventricular fibrillation which is one of phenomena caused by abnormality of the heart, the QRA waveform is irregular (see Non Patent Literature 2).

In the third-type cardiac state estimation processing, the first integrated estimation processing is performed after the first-type cardiac state estimation processing and the second-type cardiac state estimation processing are performed. In the first integrated estimation processing, the state of the heart of the estimation target 9 is estimated to be abnormal only in a case where both the first-type cardiac state estimation processing and the second-type cardiac state estimation processing estimate that the state of the heart of the estimation target 9 is abnormal.

Therefore, in the first integrated estimation processing, for example, the state of the heart of the estimation target 9 is estimated to be normal in a case where the estimation result by the execution of the second-type cardiac state estimation processing estimates that the state of the heart is normal even in a case where the state of the heart is estimated to be abnormal by the execution of the first-type cardiac state estimation processing.

As described above, the third-type cardiac state estimation processing is processing of estimating the state of the heart of the estimation target 9 using not only the estimation result of either the first-type cardiac state estimation processing or the second-type cardiac state estimation processing but also the estimation results of both the first-type cardiac state estimation processing and the second-type cardiac state estimation processing.

Therefore, the abnormal state estimation system 100 of the second modification configured as described above can estimate the state of the heart of the estimation target 9 with higher accuracy than a case where the state of the heart of the estimation target 9 is estimated using the estimation result of either the first-type cardiac state estimation processing or the second-type cardiac state estimation processing. That is, the abnormal state estimation system 100 of the second modification estimates the state of the heart of the estimation target 9 based on the two conditions of the generation of the signal and the irregularity of the QRS waveform in the non-responsive period, so that the state of the heart of the estimation target 9 can be estimated with high accuracy.

Note that the generation of the signal in the non-responsive period means that the peak period appearance condition is satisfied.

Note that, in the second-type cardiac state estimation processing executed in the third-type cardiac state estimation processing, it is not always necessary to estimate the state of the heart of the estimation target 9 depending on whether or not the RRI exceeds the threshold value, and the state of the heart of the estimation target 9 may be estimated using the statistic of the RRI. That is, the state of the heart of the estimation target 9 may be estimated by estimating the irregularity of the QRS waveform in the ventricular fibrillation using the statistic of the RRI as the irregularity of the RRI.

The statistic of RRI may be, for example, an average of RRI, a deviation, a variance value, a median value, an absolute deviation, a root mean square value, a percentile value, a maximum value, or a minimum value.

For example, in a case where the statistic of the RRI is the average of the RRI, in the third-type cardiac state estimation processing, in a case where the difference between the repeatedly calculated average values of the RRO exceeds a predetermined threshold value, when the occurrence of a signal in the non-responsive period is confirmed, the state of the heart of the estimation target 9 is estimated to be abnormal.

Note that the statistic of the RRI may be a statistic other than the average such as a deviation. Even in a case where the statistic of the RRI is another statistic, the state of the heart of the estimation target 9 is estimated to be abnormal depending on whether or not the difference in the repetitively calculated statistic exceeds a predetermined threshold value.

(Third Modification)

As the cardiac state estimation processing to be executed, the cardiac state estimation unit 420 may execute fourth-type cardiac state estimation processing instead of the first-type cardiac state estimation processing. That is, the cardiac state estimation unit 420 may estimate the state of the heart of the estimation target 9 by executing the fourth-type cardiac state estimation processing on the cardiac state time series indicated by the cardiac state signal acquired by the cardiac state time series acquisition unit 410.

<Fourth-Type Cardiac State Estimation Processing>

The fourth-type cardiac state estimation processing is processing of executing the first-type cardiac state estimation processing, the second-type cardiac state estimation processing, and the second integrated estimation processing. The second integrated estimation processing is processing of estimating the state of the heart of the estimation target 9 based on the estimation result of the first-type cardiac state estimation processing and the estimation result of the second-type cardiac state estimation processing.

The second integrated estimation processing is processing of estimating that the state of the heart of the estimation target 9 is abnormal regardless of the estimation result of the first-type cardiac state estimation processing in a case where the estimation result of the second-type cardiac state estimation processing is an abnormal result. In this respect, the first integrated estimation processing and the second integrated estimation processing are different.

The second integrated estimation processing is processing of estimating that the state of the heart of the estimation target 9 is abnormal in a case where the estimation result of the second-type cardiac state estimation processing is normal even in a case where the estimation result of the first-type cardiac state estimation processing is abnormal. The second integrated estimation processing is processing of estimating that the state of the heart of the estimation target 9 is normal in a case where the estimation result of the first-type cardiac state estimation processing is normal, and in a case where the estimation result of the second-type cardiac state estimation processing is normal.

In the fourth-type cardiac state estimation processing, the second integrated estimation processing is executed after the first-type cardiac state estimation processing and the second-type cardiac state estimation processing are executed.

As described above, the fourth-type cardiac state estimation processing is processing of estimating the state of the heart of the estimation target 9 using not only the estimation result of either the first-type cardiac state estimation processing or the second-type cardiac state estimation processing but also the estimation results of both the first-type cardiac state estimation processing and the second-type cardiac state estimation processing.

Therefore, the abnormal state estimation system 100 of the third modification configured as described above can estimate the state of the heart of the estimation target 9 with higher accuracy than in a case where the state of the heart of the estimation target 9 is estimated using the estimation result of either the first-type cardiac state estimation processing or the second-type cardiac state estimation processing.

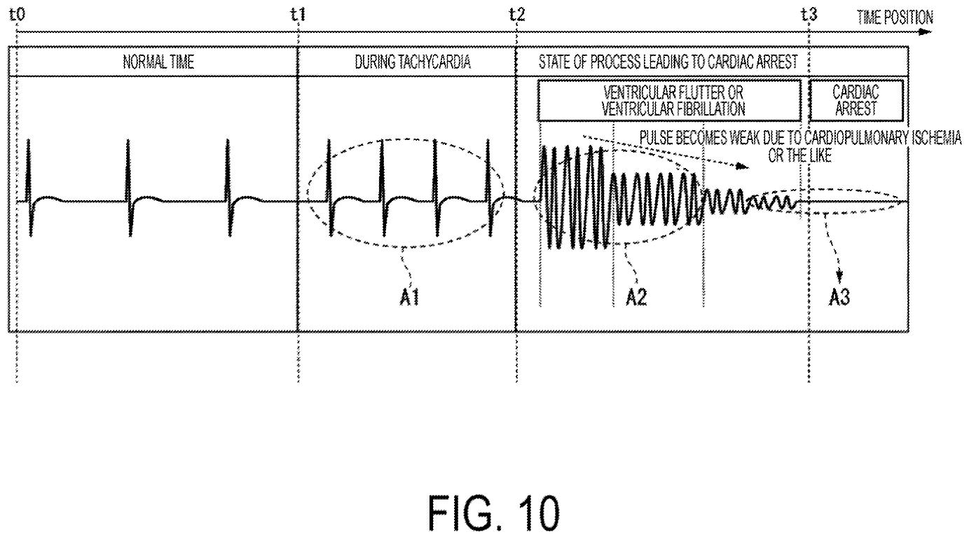

FIG. 10 is an explanatory diagram for describing an effect obtained by the fourth-type cardiac state estimation processing according to the modification. FIG. 10 illustrates a process in which ventricular fibrillation occurs after ventricular tachycardia occurs in the estimation target 9 in which a normal cardiac potential has occurred. FIG. 10 illustrates that the heart is normal in the period from time position t0 to time position t1. FIG. 10 illustrates that tachycardia occurs in the period from time position t1 to time position t2. FIG. 10 illustrates that the ventricular flutter or the ventricular fibrillation occurs in the period from time position t2 to time position t3. The waveform in the period from time t2 to time t3 in FIG. 10 is an example of a waveform indicating that the pulse becomes weak due to, for example, cardiopulmonary ischemia or the like. FIG. 10 illustrates transition to the state of cardiac arrest after time position t3.

A waveform surrounded by a frame A1 in FIG. 10 is an example of a waveform estimated to be abnormal by the second-type cardiac state estimation processing. A waveform surrounded by a frame A2 in FIG. 10 is an example of a waveform estimated to be abnormal by the first-type cardiac state estimation process. A waveform surrounded by a region A3 in FIG. 10 is an example of a waveform leading to cardiac arrest.

(Fourth Modification)

As the cardiac state estimation processing to be executed, the cardiac state estimation unit 420 may execute fifth-type cardiac state estimation processing instead of the first-type cardiac state estimation processing. That is, the cardiac state estimation unit 420 may estimate the state of the heart of the estimation target 9 by executing the fifth-type cardiac state estimation processing on the cardiac state time series indicated by the cardiac state signal acquired by the cardiac state time series acquisition unit 410.

<Fifth-Type Cardiac State Estimation Processing>

The fifth-type cardiac state estimation processing is different from the first-type cardiac state estimation processing to the fourth-type cardiac state estimation processing in that a state of cardiac arrest is estimated. The state of the cardiac arrest is, for example, a state after time t3 in FIG. 10. The fifth-type cardiac state estimation processing is processing of executing the first-type cardiac state estimation processing, the second-type cardiac state estimation processing, the first integrated estimation processing, the second integrated estimation processing, and cardiac arrest estimation processing. In the fifth-type cardiac state estimation processing, the first integrated estimation processing and the second integrated estimation processing are executed after the execution of the first-type cardiac state estimation processing and the second-type cardiac state estimation processing, and then the cardiac arrest estimation processing is executed.

The cardiac arrest estimation processing is processing of estimating that the state of the heart of the estimation target 9 is the state of cardiac arrest in a case where the deviation of the distribution of the cardiac state quantity within a predetermined period after the abnormality occurrence time position is equal to or less than a predetermined threshold value. The abnormality occurrence time position is a time position at which the state of the heart of the estimation target 9 is estimated to be abnormal by the first integrated estimation processing or the second integrated estimation processing.

At the time of cardiac arrest, the variation of the cardiac potential associated with the heartbeat is substantially zero, and is substantially the same as 0 mV. Therefore, in the cardiac arrest estimation processing, for example, processing of calculating the deviation of the distribution of the cardiac state quantity every 200 ms and processing of determining whether or not the calculated deviation is out of the range of ±15 mV are executed.

Therefore, the abnormal state estimation system 100 of the fourth modification configured as described above can detect the cardiac arrest by executing the fifth-type cardiac state estimation processing.

(Fifth Modification)

The cardiac state estimation unit 420 may perform shaping of the waveform of the cardiac state time series using a filter that performs various types of signal processing such as an analog filter, a digital filter such as a finite impulse response (FIR) or an infinite impulse response (IR), and a moving average filter to which a moving average is applied. For example, noise components included in the cardiac state time series are removed by using the filter.

The statistic calculated in the statistic calculation processing is not limited to the average and the deviation, and may be a statistic such as a variance value, an average value, a median value, an absolute deviation, a root mean square value, a percentile value, a maximum value, or a minimum value.

With respect to the setting of the peak determination target period, a new peak determination target period from 0 milliseconds may be set every time the data is updated in accordance with the sampling rate, and each peak determination target period may be set to overlap.

The peak period appearance condition does not necessarily include a condition that the peak period is continuous. Therefore, the peak period appearance condition may be a condition that the peak period occurs 4 times or more regardless of whether the peak period is continuous or discontinuous within 1000 milliseconds, for example.

The monitoring device 4 may be implemented by using a plurality of information processing devices connected to be capable of communicating with each other via a network. In this case, the functional units included in the monitoring device 4 may be implemented in a distributed manner in the plurality of information processing devices.

The control device 5 may be implemented by using a plurality of information processing devices connected to be capable of communicating with each other via a network. In this case, the functional units included in the control device 5 may be implemented in a distributed manner in the plurality of information processing devices.

Note that the monitoring device 4 and the control device 5 are not necessarily mounted as different devices. The monitoring device 4 and the control device 5 may be implemented as one device having both functions, for example. For example, the control unit 41 may include the notification determination unit 520.

Note that all or some of the functions of the abnormal state estimation system 100 may be realized using hardware such as an application specific integrated circuit (ASIC), a programmable logic device (PLD), or a field programmable gate array (FPGA). The program may be recorded on a computer-readable recording medium. The “computer-readable recording medium” refers to, for example, a portable medium such as a flexible disk, a magneto-optical disc, a read-only memory (ROM), or a compact disc read-only memory (CD-ROM), or a storage device such as a hard disk built in a computer system. The program may be transmitted via an electrical communication line.

Note that the monitoring device 4 is an example of a state estimation device.

Although the embodiment of the present invention has been described in detail with reference to the drawings, specific configurations are not limited to the embodiment and include design and the like without departing from the gist of the present invention.

### REFERENCE SIGNS LIST

100 Abnormal state estimation system
1 Biological signal acquisition device
2 Relay terminal
3 Environmental sensor
4 Monitoring device
5 Control device
41 Control unit
42 Input unit
43 Communication unit
44 Storage unit
45 Output unit
410 Cardiac state time series acquisition unit
420 Cardiac state estimation unit
430 Storage control unit
440 Communication control unit
450 Output control unit
460 Environment information acquisition unit
51 Control unit
52 Input unit
53 Communication unit
54 Storage unit
55 Output unit
510 Estimation result acquisition unit.
520 Notification determination unit
530 Storage control unit
540 Communication control unit
550 Output control unit
91 Processor
92 Memory
93 Processor
94 Memory
9 Estimation target
90 Automobile

1. A state estimation device comprising:
a processor; and
a storage medium having computer program instructions stored thereon, wherein the computer program instruction, when executed by the processor, perform processing of:
acquiring a cardiac state time series that is a time series of a cardiac state quantity that is a quantity indicating a state of a heart as an estimation target; and
estimating a state of the heart as the estimation target based on an occurrence time of out-of-range data, using, as the out-of-range data, a non-responsive period sample whose value is outside a threshold region of processing determined according to a distribution of the non-responsive period sample among non-responsive period samples that are non-responsive period samples among samples of the cardiac state time series.

2. The state estimation device according to claim 1, wherein
estimates the state of the heart as the estimation target is also estimated based on a time interval RR-Interval (RRI) of an R wave in the cardiac state time series.

3. The state estimation device according to claim 2, wherein
the state of the heart as the estimation target is estimated as being abnormal in a case where an estimation result of the state of the heart as the estimation target based on the time interval RRI of the R wave in the cardiac state time series is an estimation result that the state of the heart as the estimation target is abnormal, and an estimation result of the state of the heart as the estimation target based on the occurrence time of the out-of-range data is also an estimation result that the state of the heart as the estimation target is abnormal.

4. The state estimation device according to claim 2, wherein
in a case where an estimation result of the state of the heart as the estimation target based on the time interval RRI of the R wave in the cardiac state time series is an estimation result that the state of the heart as the estimation target is abnormal, the the state of the heart as the estimation target is estimated as being abnormal regardless of an estimation result of the state of the heart as the estimation target based on the occurrence time of the out-of-range data.

5. The state estimation device according to claim 2, wherein
assuming a position in a time axis direction of each of the samples of the cardiac state time series as a time position, the state of the heart as the estimation target is estimated as being a state of cardiac arrest in a case where a deviation of a distribution of cardiac state quantities within a predetermined period after an abnormality occurrence time position, which is a time position at which the state of the heart as the estimation target is estimated to be abnormal by any one or both of an estimation result of the state of the heart as the estimation target based on the time interval RRI of the R wave in the cardiac state time series and an estimation result of the state of the heart as the estimation target based on the occurrence time of the out-of-range data, is equal to or less than a predetermined threshold value.

6. A state estimation device, comprising:
a processor; and
a storage medium having computer program instructions stored thereon, wherein the computer program instruction, when executed by the processor, perform processing of:
acquiring a cardiac state time series that is a time series of a cardiac state quantity that is a quantity indicating a state of a heart as an estimation target; and
estimating a state of the heart as the estimation target based on a time interval RR-Interval (RRI) of an R wave in the cardiac state time series.

7. A state estimation method, comprising:
acquiring a cardiac state time series that is a time series of a cardiac state quantity that is a quantity indicating a state of a heart as an estimation target; and
estimating a state of the heart as the estimation target based on an occurrence time of out-of-range data, using, as the out-of-range data, a non-responsive period sample whose value is outside a threshold region of processing determined according to a distribution of the non-responsive period sample among non-responsive period samples that are non-responsive period samples among samples of the cardiac state time series.

8. A non-transitory computer readable medium which stores a program for causing a computer to function as the state estimation device according to claim 1.
